(19) United States
(12) Patent Application Publication
(10) Pub. No.: US 2013/0035600 A1
(43) Pub. Date: Feb. 7, 2013

(54) METHOD TO MEASURE ARTERIAL ELASTICITY AND ARTERIOSCLEROSIS

(71) Applicant: Ching-Chuan Wei, Taichung City (TW)

(72) Inventor: Ching-Chuan Wei, Taichung City (TW)

(21) Appl. No.: 13/276,074

(22) Filed: Oct. 18, 2011

(30) Foreign Application Priority Data
Aug. 1, 2011 (TW) 100127149

(51) Int. Cl. A61B 5/02

(52) U.S. Cl. 600/481

(57) ABSTRACT

A method to measure arterial elasticity and arteriosclerosis includes a measuring step and a sampling step to obtain a pulse waveform which represents the displacement of an arterial wall during elastic dilation and contraction. The method of the invention also includes a first differentiation step and a second differentiation step to respectively obtain a first differential pulse waveform and a second differential pulse waveform. The evaluation data is obtained when the arterial wall dilates to the maximum thereof. In an evaluating step, the maximum elastic force represented by the second differentiation of the maximum displacement of the arterial wall is divided by the maximum displacement of the arterial wall to obtain an arterial elasticity coefficient to determine extent of arteriosclerosis and prevent cardiovascular diseases. The present invention has advantages of noninvasive and direct evaluation to arterial elasticity, easy operation, high accuracy and low cost and thus has very high utility.

### FIELD OF THE INVENTION

The present invention relates to a biomedical inspection method, particularly to a method to measure arterial elasticity and arteriosclerosis.

### BACKGROUND OF THE INVENTION

Owing to dainty, greasy and salty foods, modern people are likely to suffer from arteriosclerosis-related diseases, such as cerebrovascular diseases, cardiac diseases, and hypertension. Therefore, how to effectively inspect arterial elasticity and prevent the related diseases is people aim to achieve. Among approaches of the arterial elasticity inspection, noninvasive inspection is the one easier to undertake and more acceptable for people. One of the noninvasive arterial elasticity inspection methods is to capture ultrasonic images incorporating blood pressure measuring data to obtain the information such as diameter, tissue thickness and related data of blood vessels to determine the extent of arteriosclerosis. However, the above-mentioned noninvasive arterial elasticity inspection method is indirect, so that the accuracy depends on the resolution of the ultrasonic images, which further involves with the cost of image capturing equipment. It is unaffordable for ordinary people to periodically perform the inspection at home with the above-mentioned equipment.

The PWV (Pulse Wave Velocity) technology is another noninvasive arterial elasticity inspection method, which inspects the points with greater vibration and measures at least five points to obtain the average thereof, whereby to avoid bigger error in the data and achieve higher precision. The measured points include the blood vessel in the inner side of the thigh. As the inner side of the thigh is not a position convenient to measure, the testee has to wear loose clothes. Besides, the inner side of the thigh also is a private position where is not everyone could accept.

Another inspection method is to use a vibrator to vibrate the skin of the testee with different frequencies and use a force sensor to measure the reaction due to the vibration to obtain arterial data and determine extent of arteriosclerosis. However, this method does not consider that blood circulation causes vessels to dilate and contract to perform data analysis and evaluation. Further, this method neither concerns the vibration of vessel walls generated by the blood circulation incorporating with the vibration caused by the vibrator that results in interference.

### SUMMARY OF THE INVENTION

The primary objective of the present invention is to solve the problem of inaccuracy in the conventional technologies that inspect arterial elasticity indirectly.

Another objective of the present invention is to solve the problem of poor popularization in the conventional technologies that inspect arterial elasticity and arteriosclerosis by using expensive equipment.

A further objective of the present invention is to solve the problem of difficult inspection in the conventional technologies.

To achieve the above-mentioned objectives, the present invention proposes a method to measure arterial elasticity and arteriosclerosis, which comprises steps of:

S1: measuring, wherein a sensor is used to contact a skin surface to sense the vibration of an arterial wall;
S2: sampling, wherein a time-dependent pulse waveform based on an arterial wall displacement caused by the vibration of the arterial wall is obtained, and wherein the pulse waveform has a peak defined as a maximum displacement point;
S3: performing a first differentiation, wherein the pulse waveform is differentiated to obtain a first differential pulse waveform, and wherein the first differential pulse waveform has a peak defined as a maximum dilation velocity point;
S4: performing a second differentiation, wherein the first differential pulse waveform is differentiated to obtain a second differential pulse waveform; and
S5: evaluating, wherein the value of the second differential pulse waveform corresponding to the maximum displacement point is divided by the maximum arterial wall displacement in the pulse waveform to obtain an arterial elasticity coefficient.

The present invention directly measures arterial vibration to obtain a precise pulse waveform to overcome the conventional problem of using an indirect method to calculate arterial elasticity. The present invention uses a computer and program to evaluate the first and second differential pulse waveforms to obtain the arterial elasticity coefficient. Therefore, the present invention has advantages of easy operation, low cost and high accuracy.

### BRIEF DESCRIPTION OF THE DRAWINGS

FIG. 1 is a flowchart schematically showing a method to measure arterial elasticity and arteriosclerosis according to one embodiment of the present invention;

FIG. 2 is a block diagram schematically showing the structure of a device to measure arterial elasticity and arteriosclerosis according to one embodiment of the present invention;

FIG. 3 is a diagram schematically showing a preferred position being measured according to one embodiment of the present invention;

FIG. 4 is a diagram schematically showing pulse waveform comparison according to one embodiment of the present invention;

FIG. 5A is a diagram schematically showing a pulse waveform according to one embodiment of the present invention; and

FIG. 5B is a diagram schematically showing a pulse waveform according to another embodiment of the present invention.

### DETAILED DESCRIPTION OF THE PREFERRED EMBODIMENTS

The technical contents of the present invention are described in detail in cooperation with the drawings below.

Blood circulation in a human body is not completely driven by cardiac contraction. As the vessels have elasticity, when blood passes through the vessels, the vessels can dilate and then contract to circulate the blood. Therefore, based on the Hooke's law, an arterial wall is modeled by an elastic element and a damping element for simulation according to Equation (1):

$$\frac{{\partial }^{2} P ( z , t )}{\partial {t}^{2}} + \frac{\partial P ( z , t )}{\partial t} + {v}_{0}^{2} P ( z , t ) = {V}_{∞}^{2} \frac{{\partial }^{2} P ( z , t )}{\partial {z}^{2}} \qquad ( 1 )$$

wherein P(z, t) is defined as the pressure on the arterial wall at different positions and different time points. When the position is constant, the pressure only varies with the time. Pressure variation causes the dilation and contraction of the arterial wall, i.e. the arterial wall displacement x(t). Thus, Equation (1) can be alternatively expressed by Equation (2):

$$\frac{{d}^{2} x ( t )}{d {t}^{2}} + b \frac{d x ( t )}{d t} + kx ( t ) = - {k}_{p}^{2} {V}_{∞}^{2} x ( t ) . \\ wherein \\ b \frac{d x ( t )}{d t} \qquad ( 2 )$$

is the damping force of the damping element, kx(t) is the elastic force of the elastic element, and −kₚ²V_{∞}²x(t) is the cardiac contraction force for blood circulation. The method of the present invention is performed based on Equation (2). The arterial wall displacement x(t) is the diameter variation of the vessel during dilation or contraction.

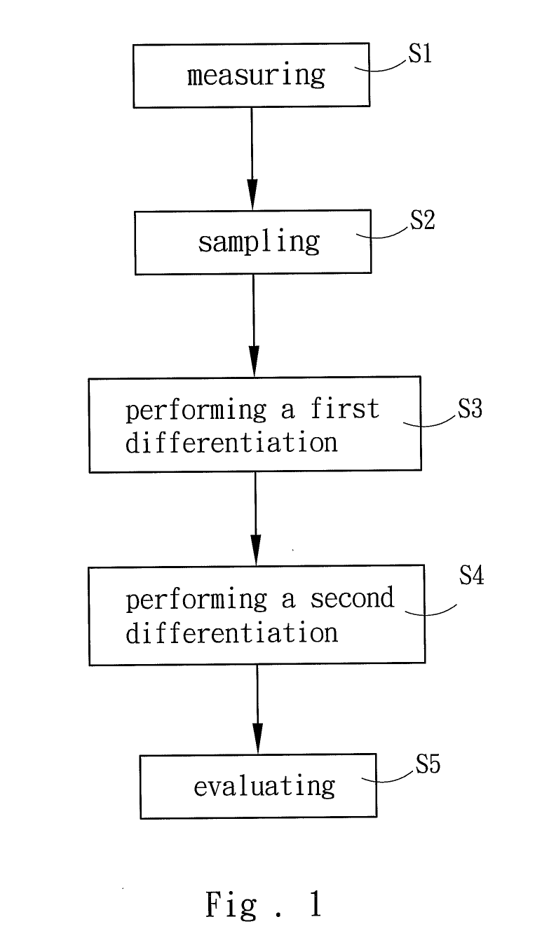

Refer to FIG. 1 a flowchart schematically showing a method to measure arterial elasticity and arteriosclerosis according to one embodiment of the present invention. The method of the present invention comprises the following steps.

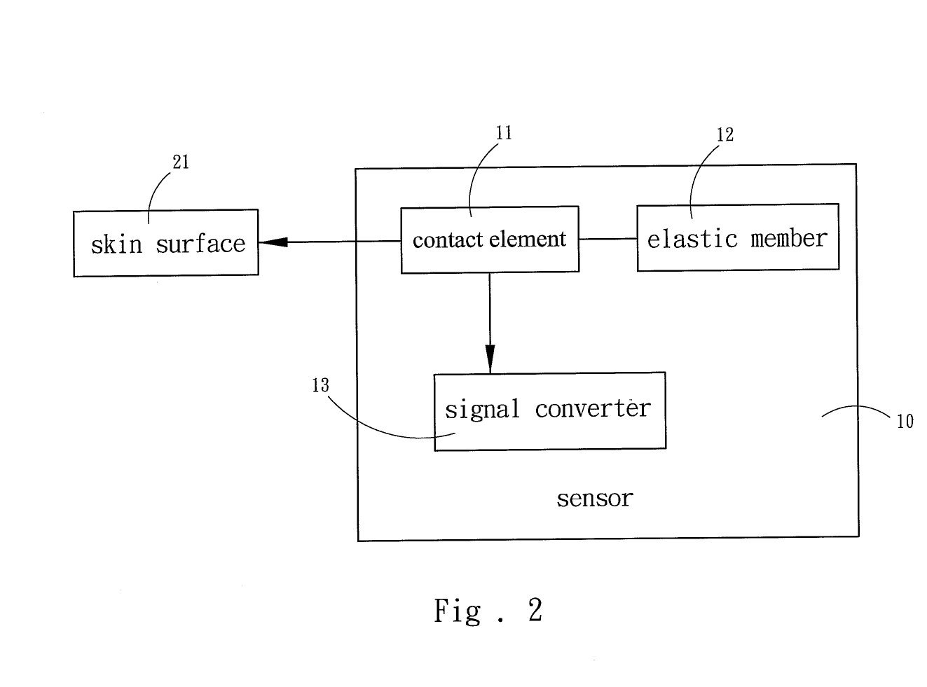

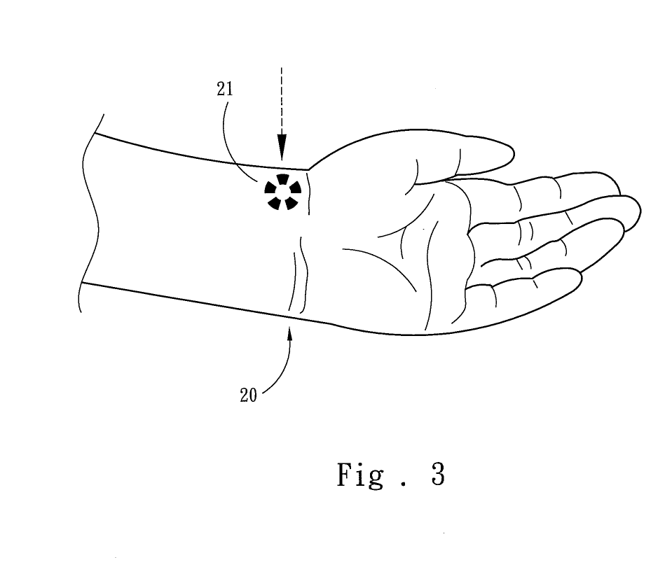

Step S1: measuring. Refer to FIG. 2 and FIG. 3. A sensor 10 is used to contact a skin surface 21 to sense vibration of an arterial wall. The sensor 10 has a contact element 11 for contacting the skin surface 21 and an elastic member 12 functioning as a buffer and connecting to the contact element 11 to enable the contact element 11 to move with vibration of the arterial wall. In one embodiment, the position where the artery appears on a wrist 20 serves as the skin contact point to sense the vibration of the arterial wall because the vibration of the artery on the wrist 20 is more obvious and easy to measure.

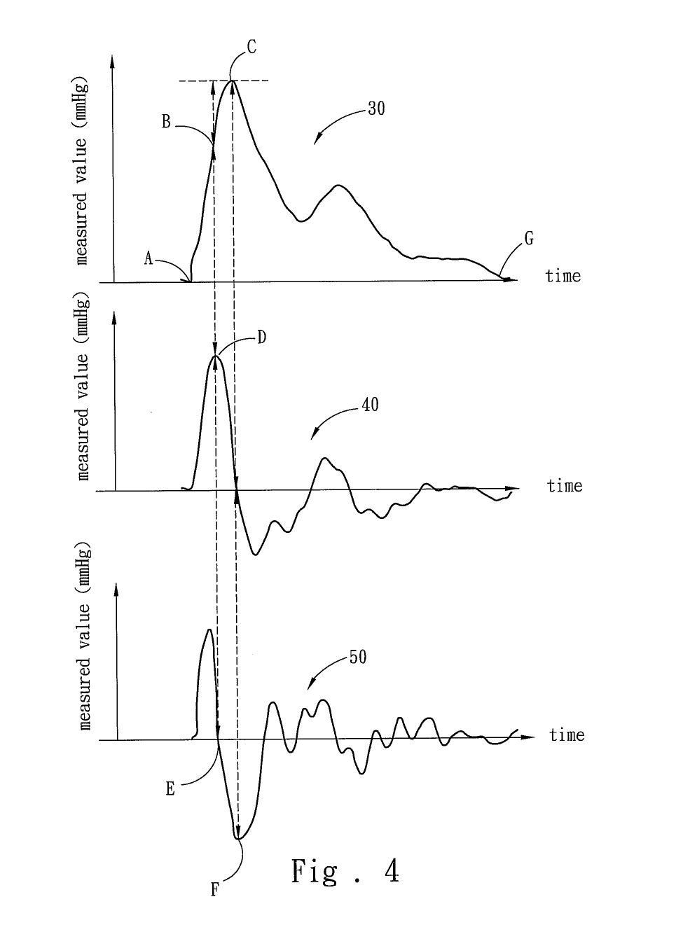

Step S2: sampling. Refer to FIG. 4. A time-dependent pulse waveform 30 according to the arterial wall displacement caused by the vibration of the arterial wall is obtained. The pulse waveform 30 has a peak defined as a maximum displacement point C. The sensor 10 has a signal converter 13 to record the displacement of the contact element 11 and convert the displacement of the contact element 11 into the pulse waveform 30. In the diagram of the pulse waveform 30, the horizontal axis represents time, and the vertical axis represents the measured value. The measured value is denoted in mmHg. The connotation of the pulse waveform 30 will be further illustrated thereinafter.

Step S3: performing a first differentiation. The pulse waveform 30 is differentiated to obtain a first differential pulse waveform 40. The first differential pulse waveform 40 has a peak defined as a maximum dilation velocity point D. The first differential pulse waveform 40 represents the displacement velocity of the arterial wall because the pulse waveform 30 is the displacement of the arterial wall. Let the maximum dilation velocity point D correspond to the time point of the pulse waveform 30 to obtain a first evaluation point B. Thud the pulse waveform 30 can be divided to a first stage from a trough A to the first evaluation point B, a second stage from the first evaluation point B to the maximum displacement point C, and a third stage from the maximum displacement point C to an end point G. In the first stage, the heart contracts and pumps blood to flow. Meanwhile, the blood flow generates reaction to dilate the arterial wall. At this time, the blood flows fastest. In the second stage, the blood flow slows down, but the arterial wall continues dilation. In the third stage, the flow velocity of blood decreases abruptly. At the maximum displacement point C, the effect of blood flow on the arterial wall can be neglected. The maximum displacement point C is the point with maximum displacement of the arterial wall, and is differentiated to become zero in the first differential pulse waveform 40. It means that the arterial wall dilates to the maximum and is going to contract at the maximum displacement point C. Therefore, neither pumping force nor damping force exists at the maximum displacement point C. Thus, Equation (2) can be modified into Equation (3):

$$\frac{{d}^{2} x ( t )}{d {t}^{2}} = - kx ( t ) {\vert}_{C} \qquad ( 3 )$$

From Equation (3), it is known that the elastic force correlates with the second differentiation of the arterial wall displacement.

Step S4: performing a second differentiation. The first differential pulse waveform 40 is differentiated to obtain a second differential pulse waveform 50. Let the maximum dilation velocity point D correspond to the time point of the second differential pulse waveform 50 to obtain a second evaluation point E. And let the maximum displacement point C correspond to the time point of the second differential pulse waveform 50 to obtain a third evaluation point F.

Step S5: evaluating. Rearrange Equation (3) to obtain Equation (4):

$$k = - \frac{\frac{{d}^{2} x ( t )}{d {t}^{2}}}{x ( t )} \qquad ( 4 )$$

From Equation (4), it is known that the arterial elasticity coefficient k correlates with the arterial wall displacement and the second differentiation of the arterial wall displacement.

In Equation (4), x(t) represents the maximum displacement of the arterial wall, i.e. the difference between the first evaluation point B and the maximum displacement point C in the vertical axis;

$$\frac{{d}^{2} x ( t )}{d {t}^{2}}$$

is the second differentiation of the maximum displacement of the arterial wall, i.e. the maximum elastic force defined by the difference between the second evaluation point E and the third evaluation point F in the vertical axis. After the maximum displacement of the arterial wall and the maximum elastic force are learned, the arterial elasticity coefficient k is obtained according to Equation (4). As the value of the second differentiation is negative, the arterial elasticity coefficient k is positive. The arterial elasticity can be learned from the arterial elasticity coefficient k to determine whether there is aging or arteriosclerosis of the vessels.

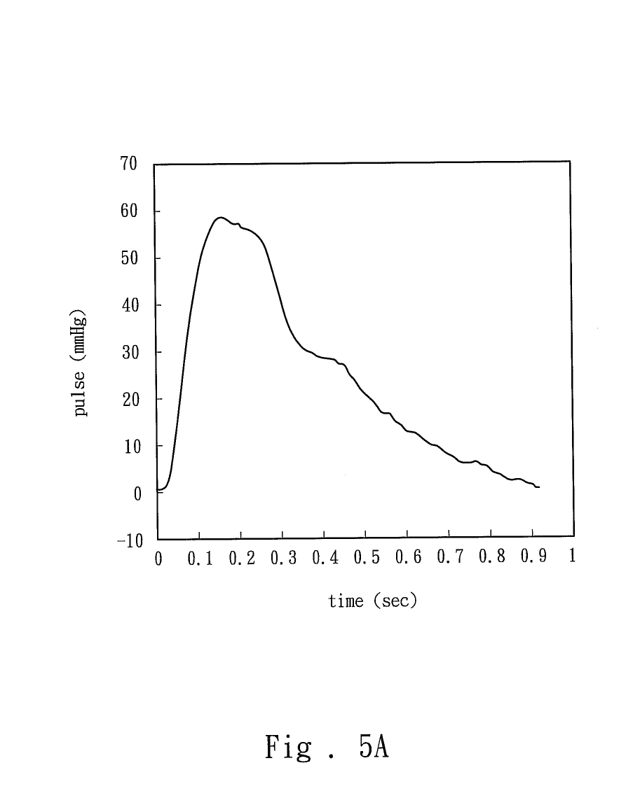

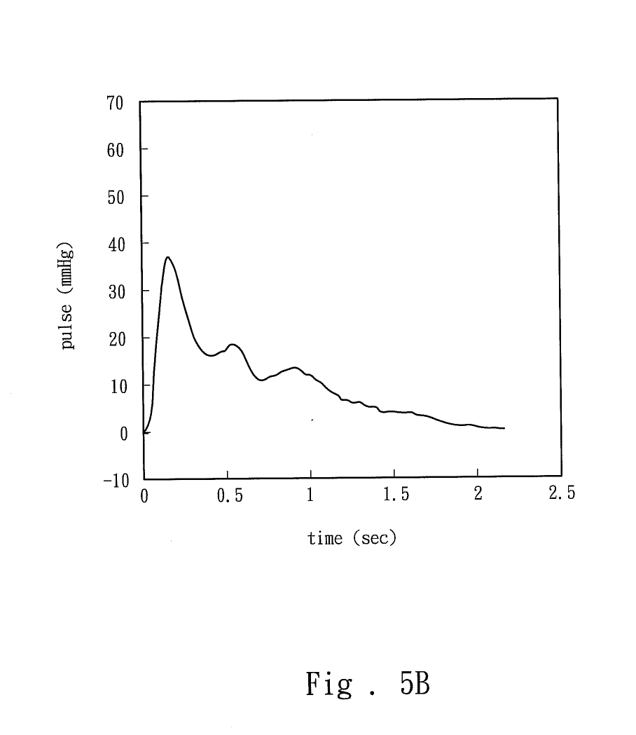

Refer to FIG. 5A for a pulse waveform of a 53-year-old male. After a second differentiation is performed, the arterial elasticity coefficient k is derived of 301.6 g/s². Refer to FIG. 5B for a pulse waveform of a 25-year-old pregnant female. After a second differentiation is performed, the arterial elasticity coefficient k is derived of 1607.0 g/s², which is much higher than that of the 53-year-old male.

In conclusion, the present invention uses the maximum displacement point C to obtain the maximum displacement of the arterial wall, and divides the value performed by secondly differentiating the maximum displacement of the arterial wall to obtain the arterial elasticity coefficient. The present invention can obtain the precise pulse waveform via merely and directly measuring the arterial vibration. The present invention also adopts the maximum displacement point C as the basic measurement point to avoid blast furnace effect and the influence of damping force, whereby is obtained an arterial elasticity coefficient with high accuracy. Thus, the present invention has advantages of easy operation, low cost and high precision. Therefore, the present invention possesses utility, novelty and non-obviousness and meets the condition for a patent.

What is claimed is:

1. A method to measure arterial elasticity and arteriosclerosis, comprising steps of:
measuring: using a sensor to contact a skin surface to sense vibration of an arterial wall;
sampling: obtaining a time-dependent pulse waveform according to an arterial wall displacement caused by the vibration of the arterial wall, wherein the pulse waveform including a peak defined as a maximum displacement point;
performing a first differentiation: differentiating the pulse waveform to obtain a first differential pulse waveform, wherein the first differential pulse waveform including a peak defined as a maximum dilation velocity point;
performing a second differentiation: differentiating the first differential pulse waveform to obtain a second differential pulse waveform; and
evaluating: dividing a value of the second differential pulse waveform at a time point corresponding to the maximum displacement point by the maximum displacement of the arterial wall in the pulse waveform to obtain an arterial elasticity coefficient.

2. The method to measure arterial elasticity and arteriosclerosis according to claim 1, wherein the pulse waveform, the first differential pulse waveform, and the second differential pulse waveform respectively have a time axis and a measured value axis vertical to the time axis.

3. The method to measure arterial elasticity and arteriosclerosis according to claim 2, wherein in the evaluating step, the maximum dilation velocity point corresponds to a time point of the pulse waveform to obtain a first evaluation point, and wherein difference between the first evaluation point and the maximum displacement point in the measured value axis is defined as maximum displacement of the arterial wall.

4. The method to measure arterial elasticity and arteriosclerosis according to claim 3, wherein in the evaluating step, the maximum dilation velocity point corresponds to a time point of the second differential pulse waveform to obtain a second evaluation point, and wherein the maximum displacement point corresponds to the time point of the second differential pulse waveform to obtain a third evaluation point, and wherein difference between the second evaluation point and the third evaluation point in the measured value axis is defined as maximum elastic force.

5. The method to measure arterial elasticity and arteriosclerosis according to claim 4, wherein the maximum elastic force is divided by the maximum displacement of the arterial wall to obtain an arterial elasticity coefficient.

6. The method to measure arterial elasticity and arteriosclerosis according to claim 1, wherein in the measuring step, the sensor includes a contact element to contact the skin surface and an elastic member functioning as a buffer and connecting to the contact element to enable the contact element to move with the vibration of the arterial wall.

7. The method to measure arterial elasticity and arteriosclerosis according to claim 6, wherein in the sampling step, the sensor includes a signal converter to record displacement of the contact element and convert the displacement of the contact element into the pulse waveform.

8. The method to measure arterial elasticity and arteriosclerosis according to claim 1, wherein in the measuring step, the sensor contacts a position where an artery appears on a wrist to sense the vibration of the arterial wall.
